(19) United States
(12) Patent Application Publication
(10) Pub. No.: US 2011/0002518 A1
(43) Pub. Date: Jan. 6, 2011

(54) METHOD AND SYSTEM FOR PROCESSING ULTRASOUND DATA

(75) Inventors: Morris Ziv-Ari, Atlit (IL); Henry Sakran, Haifa (IL); Elina Sokulin, Kiryat Tivon (IL); Alexander Sokulin, Kiryat Tivon (IL)

Correspondence Address: GE Healthcare, IP Department, 20225 Water Tower Blvd., Mail Code W492, BROOKFIELD, WI, 53045, US

(73) Assignee: General Electric Company, Schenectady, NY (US)

(21) Appl. No.: 12/496,119

(22) Filed: Jul. 1, 2009

(51) Int. Cl. A61B 8/13; A61B 8/00; G06T 7/00

(52) U.S. Cl. 382/131; 600/437; 600/453

(57) ABSTRACT

A method and system for processing ultrasound data is provided. The method includes acquiring ultrasound data and generating an image based on the ultrasound data. The method includes identifying an anatomical region of the image and automatically modifying the anatomical region for the purpose of reducing a clutter artifact. The method includes generating a modified image including at least a portion of the modified anatomical region and displaying the modified image.

### FIELD OF THE INVENTION

This disclosure relates generally to a method and system for identifying and modifying an anatomical region of an ultrasound image.

### BACKGROUND OF THE INVENTION

Conventional ultrasound systems use ultrasonic signals to determine the composition and structure of an anatomical region being studied. Typically, a transducer emits pulsed ultrasonic signals into the anatomical region and the ultrasound system determines the details about the anatomical region based on back-scattered ultrasonic signals, or echoes. By analyzing the time difference and/or any frequency shift between the transmitted ultrasonic signal and the echo, a processor within the ultrasound system is able to reconstruct various details about the anatomical region.

Images that are reconstructed from data collected with a conventional ultrasound system may experience a variety of artifacts depending upon the structure that is imaged. One of the most common artifacts in ultrasound imaging is clutter. When imaging tubular structures, such as vessels and arteries, clutter originates from the reverberation of ultrasonic signals between the walls of the tubular structure. The clutter artifact is typically a steady artifact in the image which deteriorates image quality and therefore reduces the diagnostic performance of the ultrasound system. Clutter may diminish the contrast between a vessel wall and the interior or exterior regions. This, in turn, makes it difficult to accurately localize the position of walls within tubular structures. Additionally, when color flow imaging is used to determine the blood flow within a vessel, the presence of clutter may obscure information within the color flow image.

Thus, clutter is a common artifact for ultrasound imaging. Ultrasound images that exhibit significant clutter artifacts suffer from reduced image quality for the reasons discussed hereinabove and are therefore less diagnostically useful. Therefore, there is a need for a technique to reduce clutter artifacts in ultrasound images.

### BRIEF DESCRIPTION OF THE INVENTION

The above-mentioned shortcomings, disadvantages and problems are addressed herein which will be understood by reading and understanding the following specification.

In an embodiment, a method for processing ultrasound data includes acquiring ultrasound data and generating an image based on the ultrasound data. The method includes identifying an anatomical region of the image. The method includes automatically modifying the anatomical region for the purpose of reducing a clutter artifact. The method includes generating a modified image including at least a portion of the modified anatomical region and displaying the modified image.

In an embodiment, a method for processing ultrasound data includes acquiring RF ultrasound data and demodulating the RF ultrasound data to generate raw ultrasound data. The method includes differentiating the raw ultrasound data to generate differentiated raw ultrasound data. The method includes identifying global maxima and global minima in the differentiated raw ultrasound data. The method includes generating an image based on the raw ultrasound data. The method includes identifying an anatomical region of the image based on the global maxima and the global minima. The method includes automatically modifying the anatomical region for the purpose of reducing a clutter artifact. The method includes generating a modified image comprising at least a portion of the modified anatomical region and displaying the modified image.

In an embodiment, an ultrasound system includes a transducer, a beam-former connected to the transducer, and a processor connected to the beam-former. The processor is configured to demodulate and smooth the RF ultrasound data from the beam-former to generate raw ultrasound data. The processor is configured to differentiate the raw ultrasound data to generate differentiated raw ultrasound data. The processor is configured to identify global maxima and global minima in the differentiated raw ultrasound data. The processor is configured to identify an anatomical region based on the global maxima and the global minima. The processor is configured to generate an image based on the raw ultrasound data and to modify the anatomical region of the image to generate a modified image.

Various other features, objects, and advantages of the invention will be made apparent to those skilled in the art from the accompanying drawings and detailed description thereof.

### BRIEF DESCRIPTION OF THE DRAWINGS

FIG. 1 is a schematic diagram illustrating an ultrasound system in accordance with an embodiment;

FIG. 2 is a flow chart in accordance with an embodiment; and

FIG. 3 is a graph of differentiated raw ultrasound data in accordance with an embodiment.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

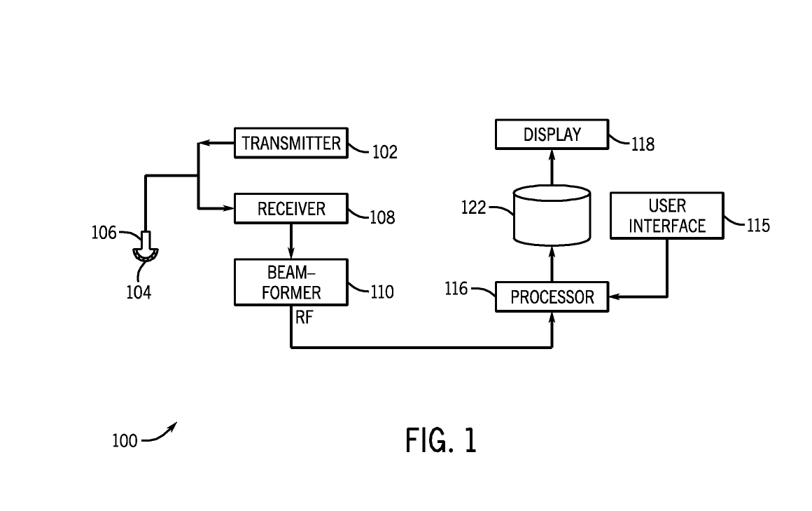

FIG. 1 is a schematic diagram of an ultrasound system 100. The ultrasound system 100 includes a transmitter 102 that drives transducers 104 within a probe 106 to emit pulsed ultrasonic signals into a body. A variety of geometries may be used. The pulsed ultrasonic signals are back-scattered from structures in the body, like blood cells or muscular tissue, to produce echoes that return to the transducers 104. The echoes are converted into electrical signals, or ultrasound data, by the transducers 104 and the electrical signals are received by a receiver 108. For purposes of this disclosure, the term ultrasound data may include data that was acquired and/or processed by an ultrasound system. Additionally, the term ultrasound data is defined to include both RF ultrasound data and raw ultrasound data, which will be discussed in detail hereinafter. The electrical signals representing the received echoes are passed through a beam-former 110 that outputs RF ultrasound data. A user interface 115 as described in more detail below may be used to control operation of the ultrasound system 100, including, to control the input of patient data, to change a scanning or display parameter, and the like.

The ultrasound system 100 also includes a processor 116 to process the ultrasound data and prepare frames of ultrasound information for display on a display 118. According to an embodiment, the processor 116 may also include a complex demodulator (not shown) that demodulates the RF ultrasound data and generates raw ultrasound data. For the purposes of this disclosure, the term “raw ultrasound data” is defined to include demodulated ultrasound data that has not yet been processed for display as an image. The processor 116 is adapted to perform one or more processing operations according to a plurality of selectable ultrasound modalities on the ultrasound information. The ultrasound information may be processed in real-time during a scanning session as the echo signals are received. For the purposes of this disclosure, the term “real-time” is defined to include a procedure that is performed without any intentional delay. Additionally or alternatively, the ultrasound information may be stored temporarily in a buffer (not shown) during a scanning session and processed in less than real-time in a live or off-line operation. Some embodiments of the invention may include multiple processors (not shown) to handle the processing tasks. For example, a first processor may be utilized to demodulate and decimate the RF signal while a second processor may be used to further process the data prior to displaying an image. It should be appreciated that other embodiments may use a different arrangement of processors.

The ultrasound system 100 may continuously acquire ultrasound information at a frame rate of, for example, 20 Hz to 30 Hz. However, other embodiments may acquire ultrasound information at a different rate. For example, some embodiments may acquire ultrasound information at a frame rate of over 100 Hz depending on the intended application. A memory 122 is included for storing processed frames of acquired ultrasound information that are not scheduled to be displayed immediately. In an exemplary embodiment, the memory 122 is of sufficient capacity to store at least several seconds worth of frames of ultrasound information. The frames of ultrasound information are stored in a manner to facilitate retrieval thereof according to its order or time of acquisition. The memory 122 may comprise any known data storage medium.

Optionally, embodiments of the present invention may be implemented utilizing contrast agents. Contrast imaging generates enhanced images of anatomical structures and blood flow in a body when using ultrasound contrast agents including microbubbles. After acquiring ultrasound data while using a contrast agent, the image analysis includes separating harmonic and linear components, enhancing the harmonic component and generating an ultrasound image by utilizing the enhanced harmonic component. Separation of harmonic components from the received signals is performed using suitable filters. The use of contrast agents for ultrasound imaging is well-known by those skilled in the art and will therefore not be described in more detail.

In various embodiments of the present invention, ultrasound information may be processed by other or different mode-related modules (e.g., B-mode, Color Doppler, power Doppler, M-mode, spectral Doppler anatomical M-mode, strain, strain rate, and the like) to form 2D or 3D data sets of image frames and the like. For example, one or more modules may generate B-mode, color Doppler, power Doppler, M-mode, anatomical M-mode, strain, strain rate, spectral Doppler image frames and combinations thereof, and the like. The image frames are stored and timing information indicating a time at which the image frame was acquired in memory may be recorded with each image frame. The modules may include, for example, a scan conversion module to perform scan conversion operations to convert the image frames from Polar to Cartesian coordinates. A video processor module may be provided that reads the image frames from a memory and displays the image frames in real time while a procedure is being carried out on a patient. A video processor module may store the image frames in an image memory, from which the images are read and displayed.

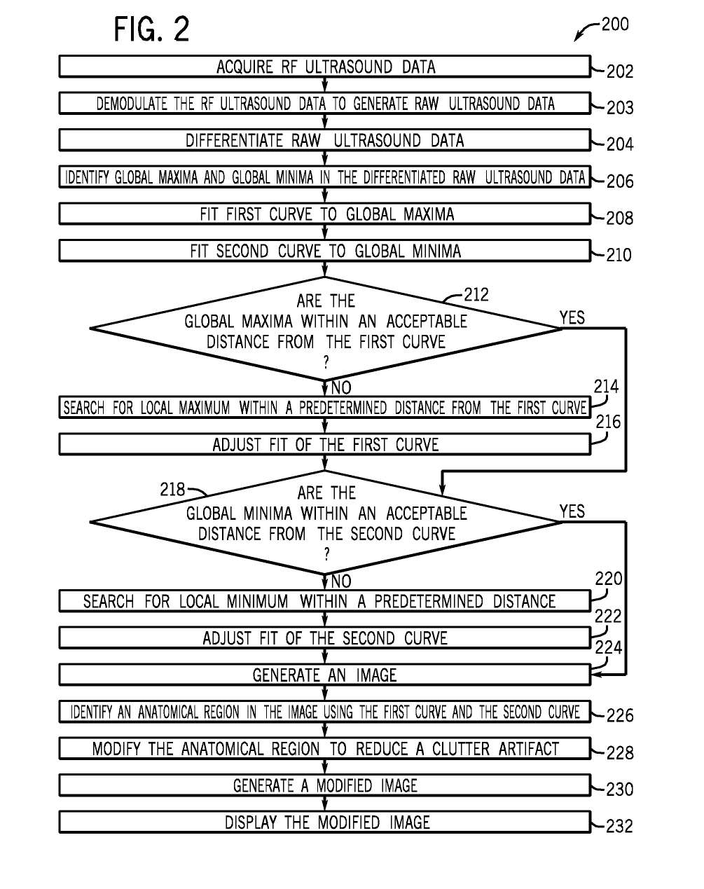

Referring to FIG. 2, a flow chart is shown in accordance with an embodiment. The individual blocks 202-232 represent steps that may be performed in accordance with the method 200. Additional embodiments may perform the steps shown in a different sequence and/or additional embodiments may include additional steps not shown in FIG. 2. The technical effect of the method 200 is the display of a modified image generated from RF ultrasound data.

Referring now to both FIGS. 1 and 2, at step 202, RF ultrasound data is acquired of an object (not shown). As was previously explained, transducers 104 within the probe 106 emit ultrasonic signals into the object. The ultrasonic signals are back-scattered and echoes are received at the transducers 104. Then, the transducers 104 convert the echoes into electrical signals, or ultrasound data, that are received at the receiver 108. The electrical signals representing the echoes are inputted into the beam-former 110, which then outputs RF ultrasound data. The RF ultrasound data may comprise data representing the echoes encoded on one or more carrier waves. According to an embodiment, the RF ultrasound data may comprise signals indicating the pressure received at each of the transducers 104 over a period of time. Next, at step 203 of FIG. 2, the processor 116 demodulates the RF ultrasound data to generate raw ultrasound data.

Still referring to FIG. 1 and FIG. 2, at step 204, the processor 116 differentiates the raw ultrasound data to form differentiated raw ultrasound data. According to one embodiment, differentiating the raw ultrasound data may comprise calculating a difference between a sample and an adjacent sample in a line of the raw ultrasound data. In this case, the sample and the adjacent sample were acquired at two different points in time, so the difference between the sample and the adjacent sample may be used to estimate a derivative at a particular sample location. Other embodiments may use alternate methods of differentiating the raw ultrasound data. For purposes of this disclosure, the term “differentiated raw ultrasound data” is defined to include data comprising derivatives or approximations of derivatives of the raw ultrasound data. According to some embodiments, the raw ultrasound data may be smoothed with a filter prior to step 204. In accordance with other embodiments, the processor 116 may differentiate the RF ultrasound data instead of the raw ultrasound data.

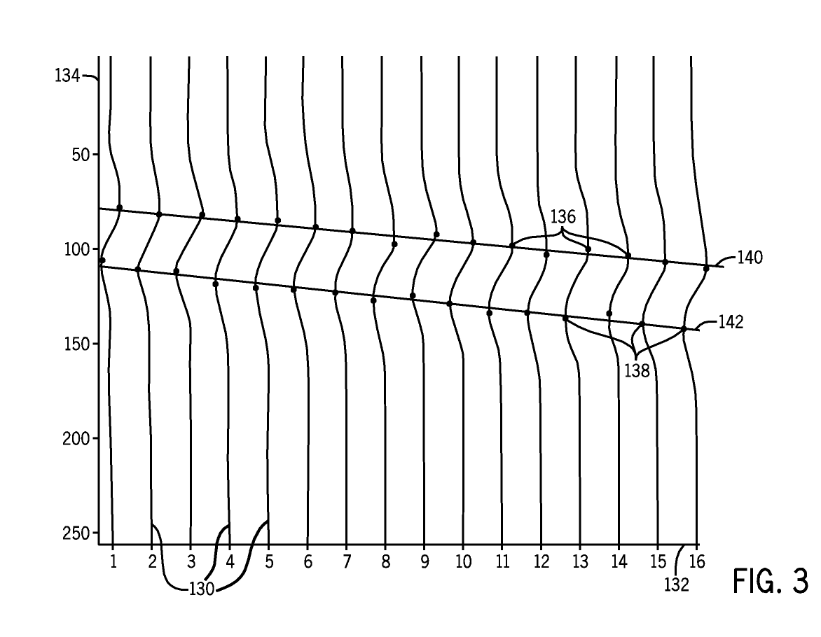

FIG. 3 is a graph of differentiated raw ultrasound data in accordance with an embodiment. The differentiated raw ultrasound data comprises a plurality of lines 130. Each of the plurality of lines 130 may represent the derivative of the vector that will ultimately be used to generate an image. According to the exemplary embodiment shown in FIG. 3, 16 lines representing the derivatives of 16 vectors are shown. It should be appreciated by those skilled in the art that the raw ultrasound data may comprise a different number of lines in other embodiments. The number of each of the plurality of lines 130 is represented along an x-axis 132. The number of the sample in each line is represented along a y-axis 134.

Referring to both FIG. 2 and FIG. 3, at step 206 the processor 116 (shown in FIG. 1) identifies global maxima 136 and global minima 138 in the differentiated raw ultrasound data. For example, the method 200 identifies a global minimum and a global maximum for each of the lines 130 in the differentiated ultrasound data in accordance with an embodiment. By identifying a global maximum for multiple lines and a global minimum for multiple lines, the method 200 identifies global maxima 136 and global minima 138. For the purposes of this disclosure, a set of more than one maximum is referred to as maxima, and a set of more than one minimum is referred to as minima. According to an embodiment, when identifying the global maxima 136, the processor 116 identifies the sample in each line of the differentiated raw ultrasound data with the maximum value. Likewise, when identifying the global minima 138, the processor 116 identifies the sample in each line of the differentiated raw ultrasound data with the minimum value. It should be appreciated by those skilled in the art that the processor 116 may not identify a maximum value or a minimum value for all of the lines represented in the differentiated raw ultrasound data. For example, the processor 116 may only identify global maxima and global minima that meet specific parameters. As a set, the global maxima 136 may represent a collection of all the samples with the greatest positive rate-of-change, and the global minima 138 may represent a collection of all the samples with the greatest negative rate-of-change.

Still referring to FIGS. 2 and 3, at step 208, the processor 116 (shown in FIG. 1) fits a first curve 140 to the global maxima 136 that were identified at step 206. According to an embodiment, fitting the first curve to the global maxima 136 may comprise performing a curve fit to minimize the difference between the first curve 140 and the global maxima 136. Fitting the first curve 140 to the global maxima 136 may also comprise fitting a line to the global maxima 136 in accordance with an embodiment. The first curve 140 may comprise a polynomial curve, an exponential curve, or other types of best-fit curves according to additional embodiments. It is necessary to use at least two of the global maxima 136 in order to fit the first curve 140 to the global maxima 136. Some embodiments may only use the global maxima 136 that fit a criterion during step 208. An example of a criterion that may be used will be discussed hereinafter. Other embodiments may use all of the global maxima 136 that were identified during step 206.

At step 210, the processor 116 (shown in FIG. 1) fits a second curve 142 to the global minima 138 that were identified at step 206. According to an embodiment, fitting the second curve 142 to the global minima 138 may comprise performing a curve fit to minimize the difference between the second curve 142 and the global minima 138. Fitting the second curve 142 to the global minima 138 may also comprise fitting a line to the global minima 138. It is necessary to use at least two of the global minima 138 in order to fit the second curve 142 to the global minima 138. Some embodiments may only use the global minima 138 that fit a criterion during step 210. Since the global maxima may correlate to the position of a first boundary of the anatomical region and the global minima may correlate to the position of a second boundary of the anatomical region, one example of a criterion involves the spacing between a global maximum and a global minimum on a given line in the differentiated raw ultrasound data. In accordance with an exemplary embodiment, the processor 116 may only use global maxima 136 and global minima 138 that are separated by a distance that would be appropriate for the spacing of the anatomical region, such as a vessel. Other embodiments may use all of the global maxima and global minima that were identified during step 206.

At step 212, the processor 116 (shown in FIG. 1) determines if the global maxima 136 are within an acceptable distance from the first curve 140. The acceptable distance may vary based on the anatomical region being targeted and/or it may be controlled by an operator through the user interface 115. If all of the global maxima 136 are within the acceptable distance, the method 200 proceeds to step 218. However, if one or more of the global maxima 136 are outside of the acceptable distance, the method 200 advances to step 214.

At step 214, the processor 116 (shown in FIG. 1) searches for a local maximum (not shown) within a predetermined distance from the first curve. The processor 116 searches for a local maximum on the same line of the differentiated raw data as the global maximum that was outside of the acceptable distance from the first curve 140. The processor 116 identifies a local maximum that is closer to the first curve 140 than the global maximum that was identified at step 206. The processor 116 may repeat the process of identifying a local maximum for each line of the differentiated raw data with a global maximum outside of the acceptable distance. Once a local maximum has been identified on each line where the global maximum was outside of the acceptable distance, the method 200 advances to step 216.

At step 216, the processor 116 (shown in FIG. 1) adjusts the fit of the first curve 140. According to an embodiment, the processor 116 replaces the global maximum that was outside of the acceptable distance with the local maximum that was identified during step 214. The processor 116 repeats this process for each line with a global maximum outside of the acceptable distance from the first curve 140. Then the processor 116 adjusts the fit of the first curve 140 using the local maximum instead of the global maximum for the one or more lines 130 where the global maximum was outside of the acceptable distance from the first curve. For the purposes of this disclosure, the term “adjusting the fit” is defined to include recalculating the fit of a curve by using a different maximum or minimum. It should be appreciated by those skilled in the art that the introduction of a different maximum or minimum may result in a shift in the position, slope, or shape of the curve. According to an embodiment, the method 200 may iteratively cycle through steps 212 to 216 in order to further adjust and refine the fit of the first curve 140. On each successive iteration, the processor 116 may check the fit of the global maxima as well as any local maxima identified at step 214 during previous iterations. According to an embodiment, the processor 116 may reduce the value of the acceptable distance during each successive iteration through steps 212 to 216 in order to progressively refine the fit of the first curve 140.

At step 218, the processor 116 (shown in FIG. 1) determines if the global minima 138 are within an acceptable distance from the second curve 142. The acceptable distance may vary based on the anatomical region being targeted for a parameter controlled by an operator. If all of the global minima 138 are within the acceptable distance, the method 200 proceeds to step 224. However, if one or more of the global minima 138 are outside of the acceptable distance, the method 200 advances to step 220.

At step 220, the processor 116 (shown in FIG. 1) searches for a local minimum (not shown) within a predetermined distance from the second curve 142. The processor 214 searches for a local minimum on the same line of the differentiated raw data as the global minimum that was outside of the acceptable distance from the second curve 142. The processor 116 identifies a local minimum that is closer to the second curve 142 than the global minimum that was identified at step 206. The processor 116 may repeat the process of identifying a local minimum for each line of the differentiated raw data with a global minimum outside of the acceptable distance. Once a local minimum has been identified on each line where the global minimum was outside of the acceptable distance, the method 200 advances to step 222.

At step 222, the processor 116 (shown in FIG. 1) adjusts the fit of the second curve 142. According to an embodiment, the processor 116 replaces the global minimum that was outside of the acceptable distance with the local minimum that was identified during step 220. Then the processor 116 adjusts the fit of the second curve 142 using the local minimum instead of the global minimum on one or more lines 130 where the global minimum was outside of the acceptable distance from the second curve 142. According to an embodiment, the method 200 may iteratively cycle through steps 218 to 222 in order to further adjust and refine the fit of the second curve 142. On each successive iteration, the processor 116 may check the fit of the global minima 138 as well as any local minima identified at step 220 during previous iterations. According to an embodiment, the processor 116 may reduce the value of the acceptable distance during each successive iteration through steps 218 to 222 in order to progressively refine the fit of the second curve 142. It should be appreciated by those skilled in the art that the processor 116 may perform steps 212-216 and steps 218-222 in a generally simultaneous manner in accordance with other embodiments.

Referring to FIG. 2, at step 224, the processor 116 (shown in FIG. 1) generates an image from the raw ultrasound data that were generated at step 203. Generating an image from raw ultrasound data is well-known by those skilled in the art and will therefore not be described in detail. According to an embodiment, the image generated at step 212 may comprise a B-mode image. However, it should be understood that that image may comprise other modes, such as color Doppler, power Doppler, M-mode, spectral Doppler anatomical M-mode, strain, strain rate, and the like. Also, it should be appreciated that the image generated at step 224 may not be displayed according to some embodiments.

Referring to FIGS. 2 and 3, at step 226, the processor 116 (shown in FIG. 1) identifies an anatomical region of the image based on the first curve 140 that was fit to the global maxima 136 and possibly some local maxima and the second curve 142 that was fit to the global minima 138 and possibly some local minima. The region may correlate to a tubular structure, such as a vessel, according to an embodiment. According to an exemplary embodiment, the processor 116 may use the first curve 140 and the second curve 142 to determine a first boundary and a second boundary in the image that was generated at step 224. The first curve 140 may be mapped to the image to generate the first boundary and the second curve 142 may be mapped to the image to generate the second boundary. For the purposes of this disclosure, the term “map” is defined to include a process of transforming a position in the raw ultrasound data or in the differentiated raw ultrasound data to a position in an image. The processor 116 may then identify the region between the first boundary and the second boundary as a vessel region according to an embodiment. It should also be appreciated that the processor 116 may not generate a graphical representation of the first curve 140 or the second curve 142. Other embodiments may involve mapping some or all of the locations selected from global maxima 136, the local maxima (not shown), the global minima 138, and the local minima (not shown). Then the processor 116 may use the mapped locations of the maxima or the minima in order to define the anatomical region. According to other embodiments, the processor 116 may identify the anatomical region based on the image instead of the raw data. For example, the processor 116 may use image processing techniques to identify an anatomical region that is likely to be affected by a clutter artifact.

Referring to FIG. 2, at step 228 the processor 116 (shown in FIG. 1) modifies the anatomical region to create a modified anatomical region. Then, the processor 116 generates a modified image including at least a portion of the modified anatomical region. According to an embodiment, the processor 116 may automatically modify the anatomical region. For the purposes of this disclosure the term “automatically” is defined to include a step or process that occurs without additional operator input. Steps 204-232 may also occur automatically. According to an exemplary embodiment, the anatomical region may represent a vessel region. Once the vessel region has been identified, the processor 116 may modify the vessel region in order to improve image quality. For example, in order to reduce the effects of a clutter artifact, the processor 116 may reduce a gain of the vessel region. By reducing the gain of the vessel region, the appearance of the clutter artifact may be greatly reduced in the image. Other embodiments may use other techniques to improve the image quality of the anatomical region. It should be appreciated that the method 200 may be used to identify regions other than vessel regions. For example, the method 200 may be used to identify other tubular structures within a patient's body or a heart region.

Referring to FIG. 2, at step 232, the modified image may be displayed on the display 118 (shown in FIG. 1). It should be appreciated that the display 118 may only show a portion of the modified image and that the processor 116 (shown in FIG. 1) may use a range of display techniques and modes, such as B-mode, Color Doppler, power Doppler, M-mode, spectral Doppler, anatomical M-mode, 3D-mode, 4D-mode, strain, and strain rate when displaying the modified image.

According to other embodiments, a different technique may be used to identify the anatomical region. For example, either Doppler ultrasound data or Color Doppler ultrasound data may be used to identify a vessel region. For example, the processor 116 (shown in FIG. 1) may use either the Doppler ultrasound data or the Color Doppler ultrasound data to identify one or more regions exhibiting movement that would be consistent with the movement of blood within a vessel region. After the vessel regions have been identified, the processor 116 would then modify the vessel region to reduce a clutter artifact in a manner similar to that described in steps 228-232 of the method 200. It should be appreciated that it may not be necessary to generate and/or display an image from the Doppler ultrasound data or the Color Doppler ultrasound data.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

We claim:

1. A method for processing ultrasound data comprising:
acquiring ultrasound data;
generating an image based on the ultrasound data;
identifying an anatomical region of the image;
automatically modifying the anatomical region for the purpose of reducing a clutter artifact;
generating a modified image comprising at least a portion of the modified anatomical region; and
displaying the modified image.

2. The method of claim 1, wherein the anatomical region comprises a vessel region.

3. The method of claim 2, wherein said identifying the anatomical region of the image comprises using Doppler ultrasound data to identify the vessel region.

4. The method of claim 3, wherein said identifying the anatomical region of the image further comprises using Color Doppler ultrasound data to identify the vessel region.

5. The method of claim 1, wherein the anatomical region comprises a heart region.

6. The method of claim 1, wherein the ultrasound data comprises RF ultrasound data or raw ultrasound data.

7. The method of claim 1, wherein said identifying the anatomical region comprises identifying the anatomical region based on the image.

8. The method of claim 1, wherein said automatically modifying the anatomical region comprises reducing a gain in the anatomical region.

9. A method for processing ultrasound data comprising:
acquiring RF ultrasound data;
demodulating the RF ultrasound data to generate raw ultrasound data;
differentiating the raw ultrasound data to generate differentiated raw ultrasound data;
identifying global maxima and global minima in the differentiated raw ultrasound data;
generating an image based on the raw ultrasound data;
identifying an anatomical region of the image based on the global maxima and the global minima;
automatically modifying the anatomical region for the purpose of reducing a clutter artifact;
generating a modified image comprising at least a portion of the modified anatomical region; and
displaying the modified image.

10. The method of claim 9, wherein said identifying the anatomical region comprises fitting a first curve to the global maxima.

11. The method of claim 10, wherein said identifying the anatomical region further comprises fitting a second curve to the global minima.

12. The method of claim 11, wherein said identifying the anatomical region further comprises identifying the anatomical region based on the first curve and the second curve.

13. The method of claim 10, wherein said identifying the anatomical region further comprises using the first curve to identify a local maximum.

14. The method of claim 13, wherein said identifying the anatomical region further comprises adjusting the fit of the first curve based on the local maximum and identifying the anatomical region based on the adjusted first curve.

15. The method of claim 11, wherein said identifying the anatomical region further comprises using the second curve to identify a local minimum.

16. The method of claim 15, wherein said identifying the anatomical region further comprises adjusting the fit of the second curve based on the local minimum and identifying the anatomical region based on the adjusted second curve.

17. An ultrasound system comprising:
a transducer;
a beam-former connected to the transducer; and
a processor connected to the beam-former, said processor configured to:
demodulate and smooth RF ultrasound data from the beam-former to generate raw ultrasound data;
differentiate the raw ultrasound data to generate differentiated raw ultrasound data;
identify global maxima and global minima in the differentiated raw ultrasound data;
identify an anatomical region based on the global maxima and the global minima;
generate an image based on the raw ultrasound data; and
modify the anatomical region of the image to generate a modified image.

18. The ultrasound system of claim 17, wherein the processor is further configured to fit a first curve to the global maxima.

19. The ultrasound system of claim 18, wherein the processor is further configured to fit a second curve to the global minima.

20. The ultrasound system of claim 19, wherein the processor is further configured to identify at least one of a first boundary of the anatomical region based on the first curve and a second boundary of the anatomical region based on the second curve.

21. The ultrasound system of claim 17, wherein the processor is further configured to generate the modified image in real-time.

22. The ultrasound system of claim 17, wherein the processor is further configured to display the modified image.
